(19) United States
(12) Patent Application Publication
(10) Pub. No.: US 2026/0232225 A1
(43) Pub. Date: Aug. 13, 2026

(54) INFORMATION ACQUISITION DEVICE, LEARNING DEVICE, AND INFORMATION ACQUISITION METHOD

(71) Applicant: Mitsubishi Electric Corporation, Tokyo (JP)

(72) Inventors: Kento TANAKA, Tokyo (JP); Yudai NAKAMURA, Tokyo (JP)

(73) Assignee: Mitsubishi Electric Corporation, Tokyo (JP)

(21) Appl. No.: 19/152,641

(22) PCT Filed: Mar. 31, 2023
(86) PCT No.: PCT/JP2023/013458

§ 371 Date: Jul. 31, 2025

(51) Int. Cl. A61B 5/11; G06V 10/778

(52) U.S. Cl. CPC A61B 5/1128; A61B 5/1103; G06V 10/778

(57) ABSTRACT

An information acquisition device includes: an imaging information acquisition unit to acquire imaging information of a test subject placed on a mounting surface; and an awakening information acquisition unit to acquire information regarding an awakening level of the test subject placed on the mounting surface, in which the imaging information acquisition unit acquires the imaging information including a face of the test subject placed on the mounting surface that moves between a first position where an angle between the mounting surface and a horizontal plane is a first angle and a second position where the angle between the mounting surface and the horizontal plane is a second angle.

### TECHNICAL FIELD

The present disclosure relates to an information acquisition device, a learning device, and an information acquisition method.

### BACKGROUND ART

Conventionally, a device that determines an awakening level of a user using a trained model that is trained with biological information and sign data of the biological information as training data has been disclosed (see Patent Literature 1).

### CITATION LIST

### Patent Literature

Patent Literature 1: JP 2021-033748 A

### SUMMARY OF INVENTION

### Technical Problem

In general, it is desirable that the trained model used in the device as described in Patent Literature 1 is trained using a sufficient amount of training data. Thus, there is a demand for a device capable of efficiently collecting information on the awakening level of a test subject.

The present disclosure solves the above problem, and an object thereof is to provide an information acquisition device, a learning device, and an information acquisition method capable of collecting information regarding an awakening level of a test subject.

### Solution To Problem

An information acquisition device according to the present disclosure includes: an imaging information acquisition unit to acquire imaging information of a test subject placed on a mounting surface; and an awakening information acquisition unit to acquire information regarding an awakening level of the test subject placed on the mounting surface, wherein the imaging information acquisition unit acquires the imaging information including a face of the test subject placed on the mounting surface that moves between a first position where an angle between the mounting surface and a horizontal plane is a first angle and a second position where the angle between the mounting surface and the horizontal plane is a second angle.

### Advantageous Effects of Invention

According to the present disclosure, information regarding an awakening level of a test subject can be collected.

### BRIEF DESCRIPTION OF DRAWINGS

FIG. 1 is a side view illustrating an information collecting system according to a first embodiment.

FIG. 2 is a side view illustrating the information collecting system according to the first embodiment.

FIG. 3 is a block diagram illustrating the information collecting system according to the first embodiment.

FIG. 4 is a block diagram illustrating an example of a hardware configuration of an information collecting device according to the first embodiment.

FIG. 5 is a block diagram illustrating an example of a hardware configuration of the information collecting device according to the first embodiment.

FIG. 6 is a flowchart illustrating a procedure for collecting information using the information collecting system according to the first embodiment.

FIG. 7 is a block diagram illustrating a physical condition estimating system according to a second embodiment.

FIG. 8 is a schematic diagram illustrating a neural network used in a learning unit according to the second embodiment.

FIG. 9 is a flowchart illustrating processing related to generation of a trained model performed by the physical condition estimating device according to the second embodiment.

FIG. 10 is a flowchart illustrating processing related to estimation of an awakening level performed by the physical condition estimating device according to the second embodiment.

FIG. 11 is a block diagram illustrating a physical condition estimating system according to a third embodiment.

FIG. 12 is a flowchart illustrating processing related to setting of a threshold performed by the physical condition estimating device according to the third embodiment.

FIG. 13 is a flowchart illustrating processing related to estimation of an awakening level performed by the physical condition estimating device according to the third embodiment.

### DESCRIPTION OF EMBODIMENTS

Hereinafter, embodiments according to the present disclosure will be described in detail with reference to the drawings.

### First Embodiment

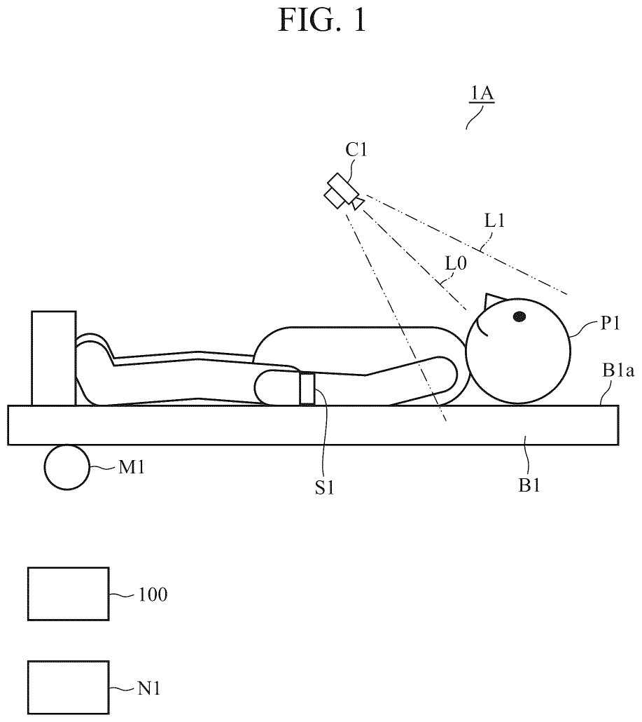

First, an information collecting system 1A according to the first embodiment will be described with reference to FIG. 1. FIG. 1 is a side view illustrating the information collecting system 1A according to the first embodiment. The information collecting system 1A is a system for collecting information including biological information from a test subject. As illustrated in FIG. 1, the information collecting system 1A according to the first embodiment includes a mounting table B1 on which a test subject P1 is placed, an imaging unit C1 that images the test subject P1, a sensor S1 for acquiring biological information of the test subject P1, a drive unit M1 that drives the mounting table B1, an input unit N1, and an information collecting device 100.

The mounting table B1 has a mounting surface B1a on which the test subject P1 is placed. For example, as illustrated in FIG. 1, the mounting table B1 is configured by a bed, and is configured to be able to mount the test subject P1 in a state in which the test subject P1 is lying on the mounting surface B1a in a state in which the planar mounting surface B1a and a horizontal plane are substantially parallel. Further, the mounting table B1 is supported so as to be rotatable about a rotation axis (not illustrated) arranged along the mounting surface B1a. Note that, in the first embodiment, a position of the mounting surface B1a where the mounting surface B1a and the horizontal plane are substantially parallel is also referred to as a first position. Further, in the first embodiment, a position of the mounting surface B1a where the mounting surface B1a intersects the horizontal plane is also referred to as a second position.

The imaging unit C1 images the test subject P1 placed on the mounting surface B1a. For example, the imaging unit C1 includes a camera having an image sensor such as a charge coupled device (CCD) or a complementary metal-oxide-semiconductor (CMOS). For example, the imaging unit C1 is held on the mounting table B1 in such a manner that the position with respect to the mounting surface B1a is fixed. Thus, the imaging unit C1 can image a specific range of the mounting surface B1a regardless of a rotation position of the mounting table B1. In other words, the imaging unit C1 can image a specific range in such a manner that the relative position between the mounting surface B1a and a center line L0 of the imaging range is constant regardless of the rotation position of the mounting table B1. For example, the imaging unit C1 is disposed in such a manner that the face of the test subject P1 placed on the mounting table B1 is included in an imaging range L1 regardless of the rotation position of the mounting table B1. In other words, the imaging unit C1 acquires imaging information of the test subject P1 placed on the mounting surface B1a in such a manner that imaging information acquired in a state where the mounting surface B1a is located at the second position and imaging information acquired in a state where the mounting surface B1a is located at the first position are imaging information in the same range with respect to the mounting surface B1a. Note that the imaging unit C1 may be configured to acquire the imaging information of the test subject P1 placed on the mounting surface B1a in such a manner that imaging information acquired in a state where the mounting surface B1a is located at the second position or in a state where the mounting surface is moving from the first position to the second position and imaging information acquired in a state where the mounting surface B1a is located at the first position or in a state where the mounting surface is moving from the second position to the first position are imaging information in the same range with respect to the mounting surface B1a.

Specifically, the imaging unit C1 is disposed at a position where the test subject P1 can be imaged from below (left side illustrated in FIG. 1) the test subject P1 placed on the mounting table B1 in such a manner that the face of the test subject P1 placed on the mounting table B1 is included in the imaging range L1 regardless of the rotation position of the mounting table B1. The imaging unit C1 outputs the captured imaging information. Note that the imaging unit C1 may be a visible light camera that detects visible light or an infrared camera that detects infrared rays.

The sensor S1 outputs a signal corresponding to a biological activity of the test subject P1. For example, the sensor S1 is disposed so as to be in contact with the test subject P1 placed on the mounting table B1, detects biological activity of the test subject P1, and outputs a signal corresponding to the detected biological activity as biological information of the test subject P1. Specifically, the sensor S1 detects a heartbeat of the test subject P1 and outputs a signal corresponding to the detected heartbeat as biological information. Note that the biological information of the test subject P1 output by the sensor S1 is not limited to the heartbeat, and may be, for example, an electrocardiogram waveform, blood pressure, brain waves, blood oxygen saturation (SPO2), body temperature, skin luminance, skin moisture content, or the like, may be any one of these pieces of information, or may be information including a combination of a plurality of these pieces of information. In addition, the sensor S1 may be an electrode for detecting a current flowing through the human body, a sensor for detecting infrared rays, a sensor for detecting radio waves, or a sensor using another physical phenomenon.

The drive unit M1 drives the mounting table B1 in such a manner that the mounting table B1 rotates about the rotation axis. For example, the drive unit M1 includes a motor and a decelerator, and rotates the mounting table B1 by current supply and a control signal from the information collecting device 100. In other words, the drive unit M1 drives the mounting table B1 in such a manner that the mounting surface B1a moves between the first position where the mounting surface B1a and the horizontal plane are substantially parallel and the second position where the mounting surface B1a and the horizontal plane intersect. In other words, the drive unit M1 drives the mounting table B1 in such a manner that the mounting surface B1a moves between the first position where the angle between the mounting surface B1a and the horizontal plane is a first angle and the second position where the angle between the mounting surface B1a and the horizontal plane is a second angle.

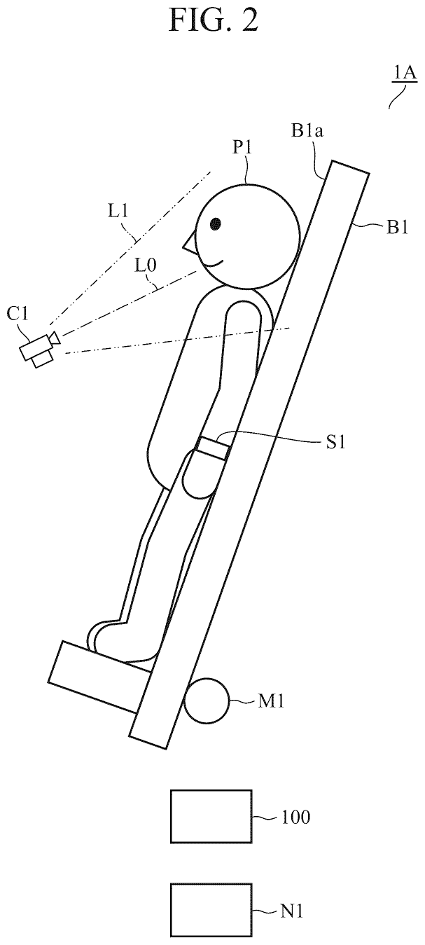

FIG. 2 is a side view illustrating the information collecting system 1A in a state where the mounting table B1 is rotated and the mounting surface B1a is located at the second position. In this state, the test subject P1 is placed on the mounting surface B1a in an upright state in which the head is positioned higher than the feet. As described above, the information collecting system 1A is configured to be capable of performing a head-up tilt test by inducing reflex syncope of the test subject P1 by changing the test subject P1 placed on the mounting table B1 from a supine state to an upright state, and observing the presence or absence of the reflex syncope.

The input unit N1 receives an input operation by an operator of the information collecting system 1A. For example, the input unit N1 receives an input operation of inputting information regarding the awakening level of the test subject P1 placed on the mounting surface B1a. Specifically, the input unit N1 receives an input operation indicating the presence or absence of a faint of the test subject P1 placed on the mounting surface B1a. For example, the input unit N1 includes an input device such as a switch, a touch panel, a keyboard, and a mouse that receive an input operation. The input unit N1 outputs a signal corresponding to the input operation. For example, when it is determined that the test subject P1 has fainted, the operator performs an input operation indicating that the test subject P1 has fainted on the input unit N1.

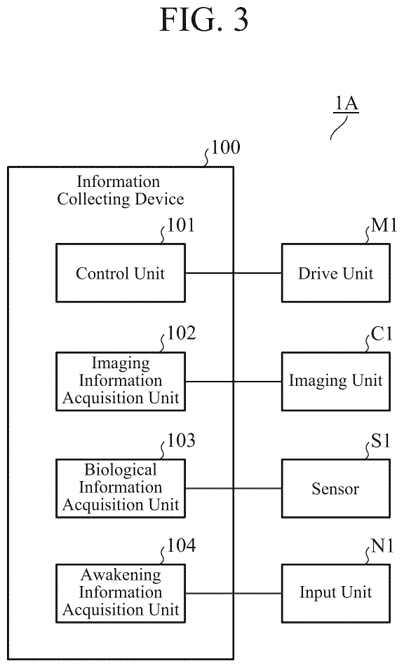

FIG. 3 is a block diagram illustrating the information collecting system 1A according to the first embodiment. As illustrated in FIG. 3, the information collecting device 100 as an information acquisition device includes a control unit 101, an imaging information acquisition unit 102, a biological information acquisition unit 103, and an awakening information acquisition unit 104, and is electrically connected to the drive unit M1, the imaging unit C1, the sensor S1, and the input unit N1. The control unit 101 controls driving of the drive unit M1 by outputting a control signal to the drive unit M1. The imaging information acquisition unit 102 acquires imaging information from the imaging unit C1. Specifically, the imaging unit C1 acquires imaging information of the test subject P1 placed on the mounting surface B1a. The biological information acquisition unit 103 acquires biological information of the test subject P1 from the sensor S1. In other words, the biological information acquisition unit 103 acquires the biological information of the test subject P1 regardless of the imaging information acquired by the imaging information acquisition unit 102. The awakening information acquisition unit 104 acquires a signal from the input unit N1. For example, the awakening information acquisition unit 104 acquires a signal corresponding to an input operation of inputting information regarding the awakening level of the test subject P1 by the operator. In other words, the awakening information acquisition unit 104 acquires the information regarding the awakening level of the test subject P1.

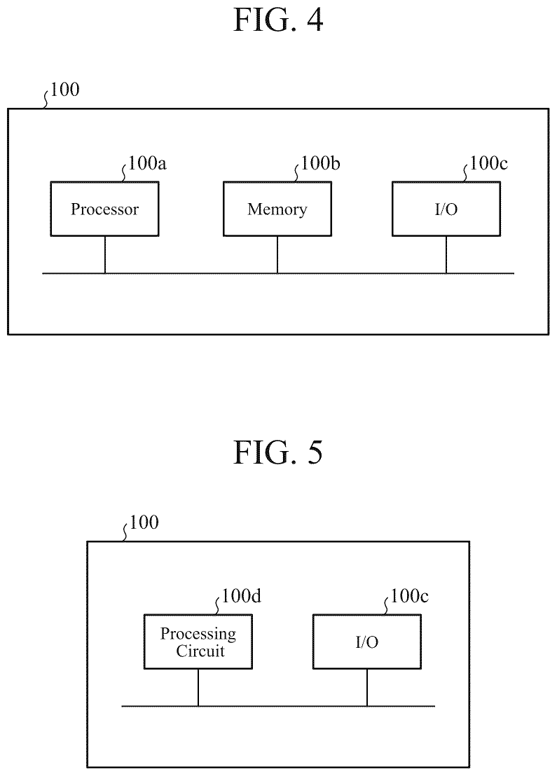

Next, a hardware configuration of the information collecting device 100 according to the first embodiment will be described with reference to FIGS. 4 and 5. FIG. 4 is a block diagram illustrating an example of a hardware configuration of the information collecting device 100 according to the first embodiment, and FIG. 5 is a block diagram illustrating an example of a hardware configuration different from that of FIG. 4 of the information collecting device 100 according to the first embodiment. For example, as illustrated in FIG. 4, the information collecting device 100 includes a processor 100a, a memory 100b, and an I/O port 100c, and is configured in such a manner that the processor 100a reads and executes a program stored in the memory 100b. The memory 100b may be, for example, a nonvolatile or volatile semiconductor memory such as RAM, ROM, a flash memory, EPROM, or EEPROM. In addition, the memory 100b may be a magnetic disk, a flexible disk, an optical disk, a compact disk, a mini disk, a DVD, or the like. Furthermore, the memory 100b may be an HDD or an SSD.

Further, for example, as illustrated in FIG. 5, the information collecting device 100 includes a processing circuit 100d and the I/O port 100c which are dedicated hardware. The processing circuit 100d includes, for example, a single circuit, a composite circuit, a programmed processor, a parallel-programmed processor, a system large-scale integration (LSI), an application specific integrated circuit (ASIC), a field programmable gate array (FPGA), or a combination thereof. Each function of the information collecting device 100 is implemented by the processor 100a or the processing circuit 100d that is dedicated hardware executing a program that is software, firmware, or a combination of software and firmware.

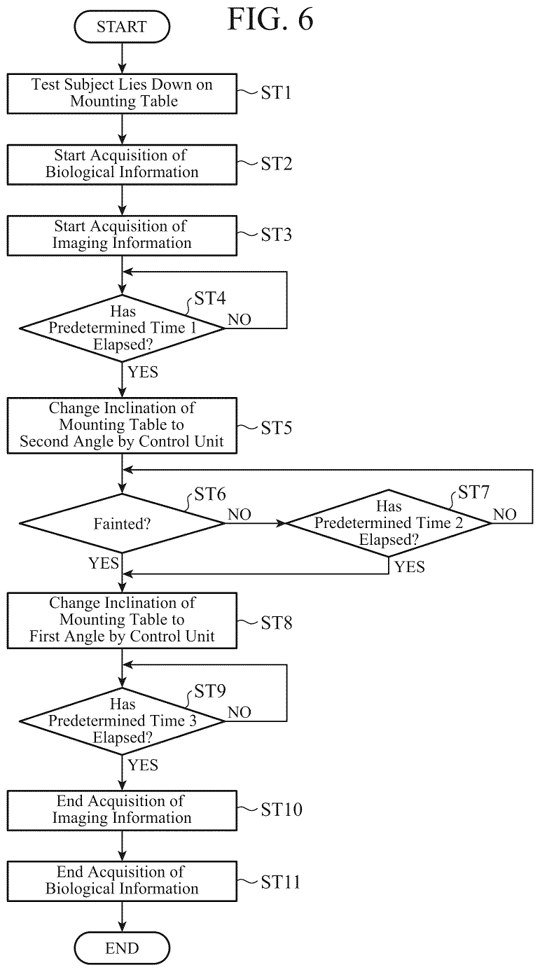

Next, a procedure for collecting information using the information collecting system 1A according to the first embodiment will be described with reference to FIG. 6. FIG. 6 is a flowchart illustrating a procedure for collecting information using the information collecting system 1A according to the first embodiment. As illustrated in FIG. 6, first, when collecting information, the operator checks that the mounting surface B1a of the mounting table B1 is in a state of being parallel to the horizontal plane, and places the test subject P1 on the mounting surface B1a so as to lie down (step ST1). In other words, the operator places the test subject P1 on the mounting surface B1a located at the first position. In this step, the operator places the test subject P1 on the mounting surface B1a in such a manner that the test subject P1 is in a posture from which information can be collected by the information collecting device 100. For example, in this step, the operator fixes the test subject P1 to the mounting surface B1a by a belt (not illustrated).

When step ST1 is performed, the operator attaches the sensor S1 to the test subject P1 and starts acquisition of biological information of the test subject P1 by the information collecting device 100 (step ST2). In this step, the information collecting device 100 starts acquisition of biological information by the sensor S1 from the test subject P1 in a state of being placed on the mounting surface B1a located at the first position. For example, the information collecting device 100 acquires biological information that changes with time from the test subject P1 by the sensor S1.

When the information collecting device 100 performs the processing of step ST2, the operator starts acquisition of imaging information of the test subject P1 by the information collecting device 100 (step ST3). In this step, the information collecting device 100 starts acquisition of imaging information of the test subject P1 imaged by the imaging unit C1. For example, in this step, the operator activates the imaging unit C1 and the information collecting device 100, and starts acquisition of imaging information of the test subject P1 by the imaging unit C1. Further, for example, the information collecting device 100 acquires imaging information of the test subject P1 as moving image information that changes with time by the imaging unit C1. Furthermore, when the processing of step ST3 is performed, the information collecting device 100 starts clocking by the control unit 101.

The information collecting device 100 determines whether or not a predetermined time 1 has elapsed after performing the processing of step ST3 (step ST4). In this processing, the control unit 101 compares the predetermined time 1 set in advance with the clocked time, and determines whether or not the time from the execution of the processing of step ST3 has exceeded the predetermined time 1. For example, the predetermined time 1 is a preset predetermined time equal to or more than 5 minutes and equal to or less than 10 minutes. When the predetermined time 1 has not elapsed after the processing in step ST3 (NO in step ST4), the information collecting device 100 waits before proceeding to the subsequent processing.

When the predetermined time 1 has elapsed since the information collecting device 100 performed the processing of step ST3 (YES in step ST4), the information collecting device 100 changes the inclination of the mounting table B1 to the second angle (step ST5). In this processing, the control unit 101 drives the drive unit M1 to rotate the mounting table B1 in such a manner that the angle between the mounting surface B1a and the horizontal plane becomes the second angle and the test subject P1 is in an upright state. For example, the second angle is a preset angle equal to or more than 60 degrees and equal to or less than 80 degrees.

When the information collecting device 100 performs step ST5, the operator observes the test subject P1 to determine whether or not the test subject P1 has fainted (step ST6). For example, in this step, the operator determines whether or not the test subject P1 has fainted on the basis of appearance observation of the test subject P1, a response of the test subject P1 to an external stimulus, and the biological information acquired by the sensor S1. For example, the operator determines whether or not the test subject P1 has fainted on the basis of weakness of the test subject P1, disappearance of a response of the test subject P1 to an external stimulus, a decrease in life activity of the test subject P1, and the like.

When it is determined in step ST6 that the test subject P1 has not fainted (NO in step ST6), in other words, when an input operation indicating a faint of the test subject P1 on the input unit N1 is not performed by the operator in step ST6, the information collecting device 100 determines whether or not a predetermined time 2 has elapsed after performing the processing of step ST3 (step ST7). In this processing, the control unit 101 compares the predetermined time 2 set in advance with the clocked time, and determines whether or not the time after performing the processing of step ST3 has exceeded the predetermined time 2. For example, the predetermined time 2 is a preset predetermined time equal to or more than 50 minutes and equal to or less than 60 minutes. When the predetermined time 2 has not elapsed after the processing in step ST3 (NO in step ST7), the information collecting device 100 returns the processing to step ST6.

When it is determined in the processing of step ST6 that the test subject P1 has fainted (YES in step ST6), and when the clocked time has passed the predetermined time 2 in the processing of step ST7 (YES in step ST7), the information collecting device 100 changes the inclination of the mounting table B1 to the first angle (step ST8). In this step, the operator performs an input operation indicating that the test subject P1 has fainted on the input unit N1. Thus, the awakening information acquisition unit 104 acquires information regarding the presence or absence of a faint of the test subject P1 placed on the mounting surface B1a. Further, in this step, the control unit 101 drives the drive unit M1 to rotate the mounting table B1 in such a manner that the angle between the mounting surface B1a and the horizontal plane becomes the first angle, that is, the test subject P1 is in a supine state. In other words, the control unit 101 drives the drive unit M1 to rotate the mounting table B1 in such a manner that the angle between the mounting surface B1a and the horizontal plane becomes the first angle, that is, the test subject P1 is in a supine state. Further, when the processing of step ST8 is performed, the information collecting device 100 starts clocking by the control unit 101.

When the information collecting device 100 performs step ST8, the information collecting device 100 determines whether or not a predetermined time 3 has elapsed after performing the processing of step ST8 (step ST8). In this processing, the control unit 101 compares the predetermined time 3 set in advance with the clocked time, and determines whether or not the time from the execution of the processing of step ST8 exceeds the predetermined time 3. For example, the predetermined time 3 is a preset predetermined time equal to or more than 5 minutes and equal to or less than 10 minutes. When the predetermined time 3 has not elapsed after the processing in step ST8 (NO in step ST9), the information collecting device 100 waits before proceeding to the subsequent processing.

When the predetermined time 3 has elapsed after the information collecting device 100 has performed the processing of step ST8 (YES in step ST9), the information collecting device 100 ends acquisition of imaging information by the imaging unit C1 (step ST10) and ends acquisition of biological information by the sensor S1 (step ST11). When the information collecting device 100 ends steps ST10 and ST11, the information collection of the test subject P1 using the information collecting system 1A ends. By performing steps ST1 to ST11 for a plurality of test subjects using the information collecting system 1A, the operator collects information available as training data for generating a trained model for determining the awakening level of the subject from imaging information of the subject for which the awakening level is to be determined.

For example, in a case where characteristic information is included in the imaging information of the test subject P1 before a faint in a case where the test subject P1 is determined to be fainted in the processing of step ST6, it is possible to generate a trained model for predicting a faint of the subject from the imaging information of the subject by generating a trained model using training data including data to which a label of a faint including the characteristic information is given and data to which a label of a non-faint not including the characteristic information is given.

Further, for example, in a case where characteristic information is included in the imaging information of the test subject P1 after a faint in a case where the test subject P1 is determined to be fainted in the processing of step ST6, it is possible to generate a trained model for determining whether or not the subject has fainted from the imaging information of the subject by generating a trained model using training data including data to which a label of a faint including the characteristic information is given and data to which a label of a non-faint not including the characteristic information is given.

Further, for example, in a case where characteristic information is included in a change in the imaging information of the test subject P1 before and after a faint in a case where the test subject P1 is determined to be fainted in the processing of step ST6, it is possible to generate a trained model for predicting a faint of the subject or determining whether or not the subject has fainted from the imaging information of the subject by generating a trained model using training data including data to which a label of a faint including the characteristic information is given and data to which a label of a non-faint not including the characteristic information is given. Note that the operator may perform steps ST1 to ST11 a plurality of times for one test subject using the information collecting system 1A to collect information available as training data for generating a trained model for determining the awakening level of the subject from the imaging information of the subject for which the awakening level is to be determined.

As described above, the information collecting system 1A according to the first embodiment includes the imaging information acquisition unit 102 to acquire imaging information of the test subject P1 placed on the mounting surface B1a, and the awakening information acquisition unit 104 to acquire information regarding the awakening level of the test subject placed on the mounting surface B1a, in which the imaging information acquisition unit 102 acquires imaging information including the face of the test subject P1 placed on the mounting surface B1a that moves between the first position where the angle between the mounting surface B1a and the horizontal plane is the first angle and the second position where the angle between the mounting surface B1a and the horizontal plane is the second angle. As described above, the information collecting system 1A can induce a change in the awakening level of the test subject P1 by changing the angle between the mounting surface B1a on which the test subject P1 is placed and the horizontal plane, and acquire the imaging information of the test subject P1 when the awakening level changes, and thus it is possible to collect information that can be used as training data for generating a trained model that determines the awakening level of the subject on the basis of the imaging information.

For example, in a vehicle such as a car including an imaging unit that images an occupant, in a case where the physical condition of the occupant is estimated using imaging information imaged by the imaging unit, it is conceivable to perform estimation using a trained model that is generated using imaging information of the occupant when the physical condition changes as training data. However, it is rare that there is a change in physical condition of the occupant, for example, a change in physical condition such as a faint, when the occupant actually gets on the vehicle, and it is difficult to collect a sufficient amount of imaging information. The information collecting system 1A according to the first embodiment can induce a change in the awakening level of the test subject in an environment simulating a state of boarding a vehicle and acquire imaging information of the test subject when the awakening level changes, without collecting imaging information of an occupant when actually boarding the vehicle.

For example, in a case where a trained model for estimating a change in the awakening level of a driver is generated on the basis of imaging information of an in-vehicle camera for imaging the driver of the vehicle, it is possible to collect information in a state where the position and the imaging range of the imaging unit C1 of the information collecting system 1A are adjusted in advance to the position and the imaging range of the in-vehicle camera of the vehicle using the trained model. The reliability of the trained model can be improved by generating the trained model using the information collected in this manner. Specifically, in a case where the in-vehicle camera of the vehicle using the trained model is disposed so as to image the driver from diagonally forward left and diagonally downward of the driver, it is possible to improve the reliability of the generated trained model by holding the imaging unit C1 of the information collecting system 1A on the mounting table B1 so as to image the test subject P1 from diagonally forward left and diagonally downward of the test subject P1 regardless of the angle between the mounting surface B1a and the horizontal plane. Further, for example, in a case where the in-vehicle camera of the vehicle using the trained model is disposed so as to image the driver from diagonally forward right and diagonally above of the driver, it is possible to improve the reliability of the generated trained model by holding the imaging unit C1 of the information collecting system 1A on the mounting table B1 so as to image the test subject P1 from diagonally forward left and diagonally downward of the test subject P1 regardless of the angle between the mounting surface B1a and the horizontal plane.

Further, the information collecting system 1A according to the first embodiment includes the biological information acquisition unit 103 that acquires the biological information of the test subject P1 by the sensor S1. Thus, for example, in a case where the feature amount regarding the biological information of the test subject P1 is extracted from the imaging information, and the trained model that determines the awakening level of the subject from the imaging information is generated using the feature amount extracted from the imaging information as the training data, it is possible to improve accuracy of the extraction of the feature amount, and improve the reliability when the feature amount is extracted from the imaging information and efficiency when creating the training data. Specifically, in a case where the feature amount regarding the heartbeat of the test subject P1 is extracted from the imaging information, and the trained model that determines the awakening level of the subject from the imaging information is generated using the feature amount extracted from the imaging information as the training data, by acquiring the biological information regarding the heartbeat of the test subject P1 by the sensor S1 together with the imaging information, it is possible to improve extraction accuracy when extracting the feature amount regarding the heartbeat of the test subject P1 from the imaging information, and to improve the efficiency when creating the training data and the reliability of the generated trained model.

Note that the biological information of the test subject P1, the feature amount of which is extracted from the imaging information, and the biological information acquired by the sensor S1 desirably include information identical or corresponding to each other, but are not limited thereto. Both the biological information of the test subject P1, the feature amount of which is extracted from the imaging information, and the biological information acquired by the sensor only need to be information that affects the awakening level of the test subject P1, for example, the biological information of the test subject P1, the feature amount of which is extracted from the imaging information, may be a change in the expression of the test subject P1, and the biological information acquired by the sensor S1 may be information related to the heartbeat of the test subject P1. Further, the information identical or corresponding to each other means information closely related to each other, and examples thereof include a heartbeat and an RR interval, an average blood pressure and a diastolic blood pressure or a systolic blood pressure, and a skin color and a body temperature.

The imaging information acquisition unit 102 acquires imaging information including the face of the test subject P1 placed on the mounting surface B1a located at least at the second position. Since the change in the awakening level including the faint of the test subject P1 in the head-up tilt test often occurs when the test subject P1 is in an upright state and often involves a change in the appearance near the face, the imaging information acquisition unit 102 acquires the imaging information including the face of the test subject P1 in an upright state, so that the imaging information including the characteristic information related to the change in the awakening level can be easily acquired, and the efficiency when creating the training data and the reliability of the generated trained model can be improved.

In the first embodiment, the information collecting device 100 is configured in such a manner that the awakening information acquisition unit 104 acquires the information indicating that the test subject P1 has fainted by an input operation on the input unit N1, but is not limited thereto. The awakening information acquisition unit only needs to be configured to acquire information related to the awakening level of the test subject P1 placed on the mounting surface B1a, and for example, the awakening information acquisition unit may be configured to acquire information indicating a state in which the consciousness has not been completely lost but the awakening level has decreased by an input operation on the input unit, or may be configured to acquire information indicating a state in which the awakening level has decreased other than faint or a state in which there is a sign of decrease of the awakening level.

Further, in the first embodiment, the information collecting device 100 is configured in such a manner that the imaging information acquisition unit 102 acquires the imaging information of the test subject P1 as moving image information that changes with time by the imaging unit C1, but is not limited thereto. The imaging information acquisition unit only needs to be configured to acquire the imaging information of the test subject P1 placed on the mounting surface B1a, and for example, the imaging information acquisition unit may be configured to acquire imaging information that is still image information at a specific single occasion (timing) from the imaging unit C1, or may be configured to acquire imaging information that is still image information at a plurality of occasions when collecting information from one test subject.

Further, in the first embodiment, the information collecting device 100 is configured in such a manner that the imaging information acquisition unit 102 acquires the imaging information of the test subject P1 from a supine state to a supine state again via an upright state by the imaging unit C1, but is not limited thereto. The imaging information acquiring unit only needs to be configured to acquire imaging information including the face of the test subject P1 placed on the mounting surface B1a at least at any occasion after the test subject P1 changes from a supine state to an upright state. In other words, the imaging information acquisition unit only needs to be configured to acquire the imaging information including the face of the test subject P1 placed on the mounting surface B1a at any occasion at least after the position of the mounting surface B1a changes from the first position to the second position. For example, the imaging information acquisition unit may start acquisition of the imaging information from the imaging unit C1 when the position of the mounting surface B1a changes from the first position to the second position, or may be configured to end acquisition of the imaging information when the position of the mounting surface B1a changes from the second position to the first position.

Further, in the first embodiment, the information collecting device 100 is configured in such a manner that the control unit 101 controls the angle between the mounting surface B1a and the horizontal plane in such a manner that the mounting surface B1a moves among the first position where the angle between the mounting surface B1a and the horizontal plane is substantially parallel, the first position where the angle between the mounting surface B1a and the horizontal plane is substantially parallel in such a manner that the test subject P1 is in a lying position, and the second position where the mounting surface B1a intersects the horizontal plane in such a manner that the test subject P1 is in an upright position, but is not limited thereto. It is sufficient if the control unit controls the rotation position of the mounting table B1 so as to induce a change in the awakening level of the test subject P1 by changing the posture of the test subject P1 placed on the mounting surface B1a, and for example, the mounting surface on which the test subject P1 is placed need not be a flat surface, and may be constituted by a plurality of surfaces intersecting each other, may be constituted by a curved surface, or may be formed by a soft material that is easily deformed by an external force. Specifically, the control unit may be configured to control the position of the mounting surface between the first position where the test subject P1 in a supine state is placed and the second position where the test subject P1 in a sitting state is placed. In addition, the mounting surface may include a first surface that comes into contact with the back of the test subject P1 in a supine state to support the test subject P1, and a second surface that comes into contact with the sole of the test subject P1 in an upright state to support the test subject P1.

Further, in the first embodiment, the information collecting device 100 is configured in such a manner that the biological information acquisition unit 103 acquires the biological information of the test subject P1 by an input signal from the sensor S1, but is not limited thereto. For example, the biological information acquisition unit may be configured to acquire the biological information of the test subject P1 by the input signal from the input unit N1, or may be configured to acquire the biological information of the test subject P1 by the sensor S1 and the input signal from the input unit N1. Specifically, the biological information acquisition unit may be configured to acquire the biological information of the test subject P1 by the operator performing an input operation on the input unit with respect to the biological information of the test subject P1 acquired by a sensor or by observation of the test subject P1 by an operator (not illustrated). Examples of the biological information of the test subject P1 acquired by the observation of the test subject P1 by the operator include characteristics of the appearance of the test subject P1 such as information regarding expression, information regarding an eye opening degree, information regarding complexion, information regarding a pupil diameter, information regarding a mouth-opening degree, and presence or absence of exhaustion. In addition, the information input from the input unit to the information collecting device 100 is not limited to the above, and may include other information regarding the test subject, for example, the age, medical history, height, weight, gender, and the like of the test subject.

Further, in the first embodiment, in the information collecting system 1A, the imaging unit C1 is held on the mounting table B1 in such a manner that the relative position between the imaging unit C1 and the mounting surface B1a becomes constant, but is not limited thereto. It is sufficient if the information collecting system 1A is configured to be able to acquire imaging information in a specific range of the mounting surface B1a regardless of the position of the mounting surface B1a, and for example, the information collecting system may include an imaging position control unit (not illustrated) that controls the position of the imaging unit in such a manner that the imaging unit moves with the rotation of the mounting table, and may be configured to be able to acquire imaging information in a specific range of the mounting surface B1a regardless of the position of the mounting surface B1a.

### Second Embodiment

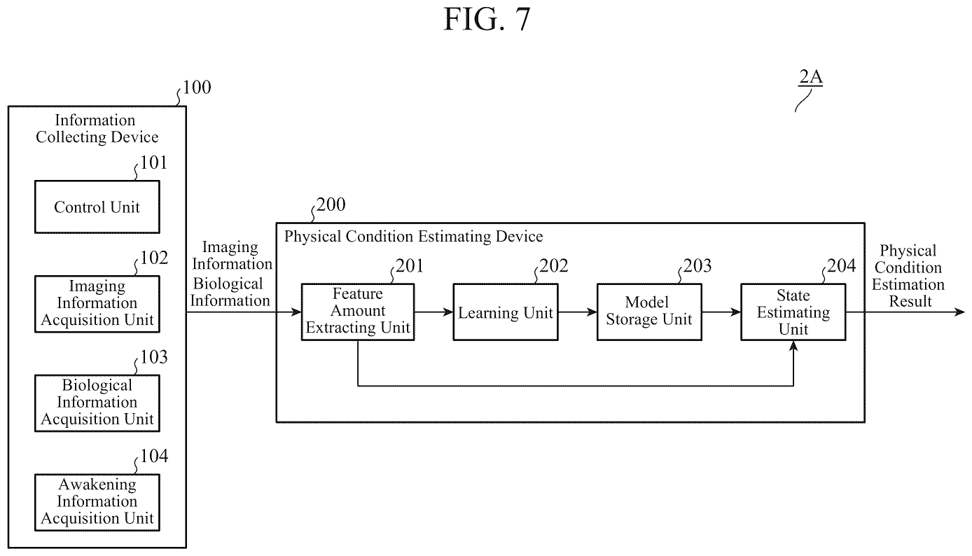

Next, a physical condition estimating system 2A according to the second embodiment will be described with reference to FIGS. 7 to 10. FIG. 7 is a block diagram illustrating the physical condition estimating system 2A according to the second embodiment. As illustrated in FIG. 7, the physical condition estimating system 2A according to the second embodiment includes the information collecting device 100 according to the first embodiment and a physical condition estimating device 200. Description of the information collecting device 100 overlapping with that of the first embodiment will be omitted.

The physical condition estimating device 200 as an awakening level estimating device and a learning device is a device that generates a trained model using information collected by the information collecting device 100 and estimates the awakening level of a subject from imaging information of the subject using the trained model. As illustrated in FIG. 7, the physical condition estimating device 200 includes a feature amount extracting unit 201, a learning unit 202, a model storage unit 203, and a state estimating unit 204.

The feature amount extracting unit 201 extracts a feature amount from imaging information and biological information of a test subject collected by the information collecting device 100. For example, the feature amount extracting unit 201 may be configured to extract one of an RR interval, a heartbeat, a diastolic blood pressure value, a systolic blood pressure value, an average blood pressure value, an eye opening degree, the mouth-opening degree, movement of expression muscles, a pupil change rate, a luminance change amount, and the like from the imaging information and the biological information of the test subject collected by the information collecting device 100, or may be configured to extract a plurality of these feature amounts.

The learning unit 202 as a trained model generating unit generates a trained model using the feature amount extracted by the feature amount extracting unit 201 and information regarding the awakening level of the test subject associated with the imaging information and the biological information from which the feature amount has been extracted as training data. As a training algorithm used by the learning unit 202 to generate the trained model, a known algorithm of supervised learning can be used. Hereinafter, a case where the learning unit 202 applies a neural network as an example of the training algorithm will be described.

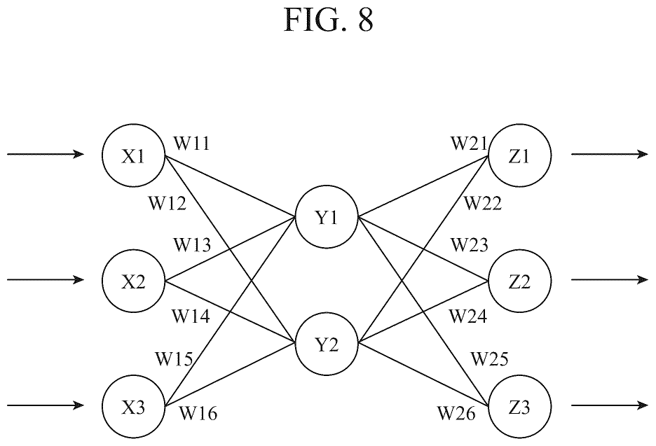

FIG. 8 is a schematic diagram illustrating a neural network used in the learning unit 202 according to the second embodiment. For example, the learning unit 202 learns the relationship between the input imaging information and the awakening level of the subject (test subject) related to the imaging information by so-called supervised learning in accordance with the neural network model. Here, the supervised learning refers to a method in which training data that is a set of input and result (label) data is given to the learning unit 202 as a learning device to learn features in the training data and infer a result from the input.

The neural network includes an input layer including a plurality of neurons, an intermediate layer (hidden layer) including a plurality of neurons, and an output layer including a plurality of neurons. The intermediate layer may be one layer or two or more layers. For example, in the case of a three-layer neural network as illustrated in FIG. 8, when a plurality of inputs is input to the input layer (X1-X3), the value is multiplied by a weight W1 (w11-w16) and input to the intermediate layer (Y1-Y2), and the result is further multiplied by a weight W2 (w21-w26) and output from the output layer (Z1-Z3).

This output result varies depending on the values of the weights W1 and W2. In the second embodiment, the neural network learns whether or not there is a state in which the awakening level of the subject is decreased or a state in which there is a sign of decrease in the awakening level by so-called supervised learning according to the training data created on the basis of the feature amount extracted by the feature amount extracting unit 201. For example, the neural network learns whether or not the subject is in a fainted state or has a faint sign according to the training data created on the basis of the feature amount extracted by the feature amount extracting unit 201. As described above, the neural network learns by adjusting the weights W1 and W2 in such a manner that the result output from the output layer after the extracted feature amount is input to the input layer approaches the feature amount indicating a decrease in the awakening level or a sign of decrease in the awakening level of the subject. The learning unit 202 generates a trained model by executing learning as described above, and stores the generated trained model in the model storage unit 203 as a storage unit.

The state estimating unit 204 as an inference unit performs inference regarding the awakening level of the subject on the basis of the trained model stored in the model storage unit 203 and imaging information of a subject for which the awakening level is to be estimated. For example, the state estimating unit 204 estimates whether or not there is a state in which the awakening level has decreased or a state in which there is a sign of decrease in the awakening level for the subject on the basis of the trained model stored in the model storage unit 203 and the imaging information of the subject for which the awakening level is to be estimated. The feature amount used for estimation by the state estimating unit 204 may be imaging information or biological information acquired from the information collecting device 100, or may be imaging information or biological information acquired from an imaging device or a biological information acquisition device (not illustrated). The state estimating unit 204 outputs the estimation result to the outside.

The physical condition estimating device 200 may include a processor, a memory, and an I/O port, and be configured in such a manner that the processor reads and executes a program stored in the memory, or may include a processing circuit and an I/O port that are dedicated hardware, and be configured in such a manner that the processing circuit executes the program. Since the hardware configuration of the physical condition estimating device 200 is similar to that of the information collecting device 100 according to the first embodiment, the description thereof will be omitted.

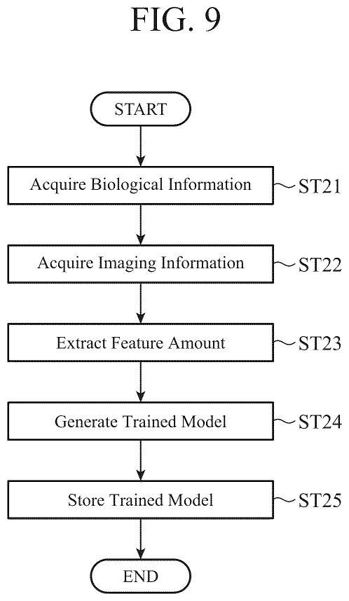

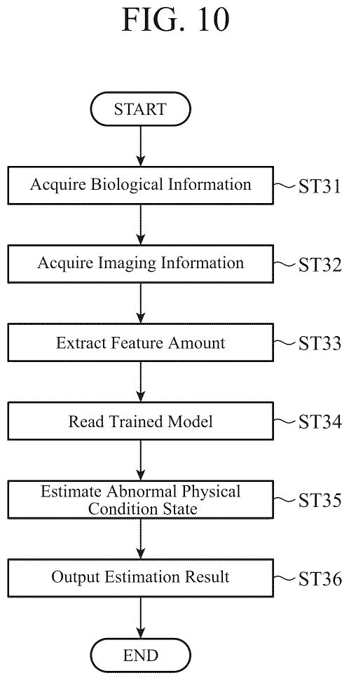

Next, processing performed by the physical condition estimating device 200 according to the second embodiment will be described with reference to FIGS. 9 and 10. FIG. 9 is a flowchart illustrating processing related to generation of a trained model performed by the physical condition estimating device 200 according to the second embodiment. First, when the processing is started, the physical condition estimating device 200 acquires the biological information collected by the information collecting device 100 (step ST21), and acquires the imaging information collected by the information collecting device 100 (step ST22). Note that the physical condition estimating device 200 may be configured to acquire information from the information collecting device 100 in a state of being electrically connected to the information collecting device 100 so as to be able to communicate information, or may be configured to acquire information from the information collecting device 100 via a recording medium.

After performing the processing of steps ST21 and ST22, the physical condition estimating device 200 extracts a feature amount from the acquired information (step ST23). Note that, in the processing of step ST23, the physical condition estimating device 200 may be configured to extract the feature amount related to the biological information of the test subject from the imaging information. For example, the physical condition estimating device 200 may be configured to extract the feature amount of the imaging information corresponding to the biological information acquired by the sensor S1 on the basis of the imaging information and the biological information acquired by the sensor S1 when the imaging information is acquired by the imaging unit C1, or may be configured to extract the feature amount related to the biological information of the test subject on the basis of a characteristic change in the appearance of the human body according to a known biological activity. The physical condition estimating device 200 can use a known algorithm when extracting the feature amount from a captured image.

When the processing of step ST23 is performed, the physical condition estimating device 200 generates a trained model on the basis of the extracted feature amount and the information regarding the awakening level of the test subject associated with the imaging information and the biological information from which the feature amount has been extracted (step ST24). In this processing, the learning unit 202 can generate the trained model using a known machine training algorithm such as the neural network as described above. After performing the processing of step ST24, the physical condition estimating device 200 stores the generated trained model in the model storage unit 203 and ends the processing (step ST25).

Next, processing related to estimation of the awakening level performed by the physical condition estimating device 200 according to the second embodiment will be described by exemplifying processing in which the physical condition estimating device 200 estimates the awakening level of the subject on the basis of imaging information and biological information acquired from the imaging device and the biological information acquisition device (not illustrated). FIG. 10 is a flowchart illustrating processing related to estimation of the awakening level performed by the physical condition estimating device 200 according to the second embodiment. Note that, in the second embodiment, the state estimating unit 204 constitutes an imaging information acquisition unit that acquires imaging information including imaging of the face of the subject.

First, when the processing is started, the physical condition estimating device 200 acquires biological information of the subject from the biological information acquisition device (not illustrated) (step ST31), and acquires imaging information including imaging of the face of the subject from the imaging device (not illustrated) (step ST32). For example, the physical condition estimating device 200 acquires the biological information and the imaging information in a state of being electrically connected to the imaging device and the biological information acquisition device (not illustrated) so as to be able to communicate information. For example, the imaging device (not illustrated) is an imaging device that images an occupant of a vehicle, and the biological information acquisition device (not illustrated) is a sensor that is provided in the vehicle and acquires biological information of the occupant.

After performing the processing of steps ST31 and ST32, the physical condition estimating device 200 extracts a feature amount from the acquired information (step ST33). In this processing, the feature amount extracting unit 201 extracts a feature amount from the acquired information according to an algorithm when the trained model is generated. For example, the feature amount extracting unit 201 extracts a feature amount regarding biological information of the test subject from the acquired imaging information.

After performing the processing of step ST33, the physical condition estimating device 200 refers to the information stored in the model storage unit 203 and reads the trained model (step ST34). Note that, in a case where the learning unit 202 is configured to generate a plurality of trained models, the physical condition estimating device 200 may be configured to select one of the trained models on the basis of the extracted feature amount.

When the processing of step ST34 is performed, the physical condition estimating device 200 estimates the awakening level of the subject by the state estimating unit 204 on the basis of the extracted feature amount and the trained model stored in the model storage unit 203 (step ST35). After performing the processing of step ST35, the physical condition estimating device 200 outputs information regarding the estimated awakening level of the subject and ends the processing (step ST36).

As described above, the physical condition estimating system 2A according to the second embodiment includes the model storage unit 203 that stores the trained model generated using the training data including the feature amount extracted from the imaging information including the imaging of the face of the test subject and the information regarding the awakening level of the test subject, and the state estimating unit 204 that acquires the imaging information including the imaging of the face of the subject, in which the state estimating unit 204 performs inference regarding the awakening level of the subject on the basis of the trained model stored in the model storage unit 203 and the acquired imaging information. Thus, it is possible to estimate the awakening level of the subject on the basis of the imaging information of the subject, and it is possible to manage the physical condition of the subject more easily than before.

Further, the physical condition estimating device 200 according to the second embodiment includes the feature amount extracting unit 201 that extracts a feature amount from the imaging information acquired by the information collecting device 100, and the learning unit 202 that generates a trained model on the basis of the information regarding the awakening level of the test subject acquired by the information collecting device 100 and the feature amount extracted by the feature amount extracting unit 201. Thus, the physical condition estimating device 200 can generate the trained model on the basis of a sufficient amount of information acquired by the information collecting device 100, and the reliability of the trained model can be improved.

Note that, in the second embodiment, the physical condition estimating system 2A is configured to generate the trained model on the basis of the biological information of the test subject acquired by the sensor or the like and the extracted feature amount, the feature amount extracted from the imaging information of the test subject, and the information regarding the awakening level of the test subject, but is not limited thereto. The physical condition estimating system only needs to be configured to generate the trained model on the basis of at least the feature amount extracted from the imaging information and the information regarding the awakening level of the test subject, and for example, the physical condition estimating system 2A may be configured not to acquire biological information not depending on the imaging information, or may be configured to estimate the awakening level of the subject without acquiring biological information not depending on the imaging information.

### Third Embodiment

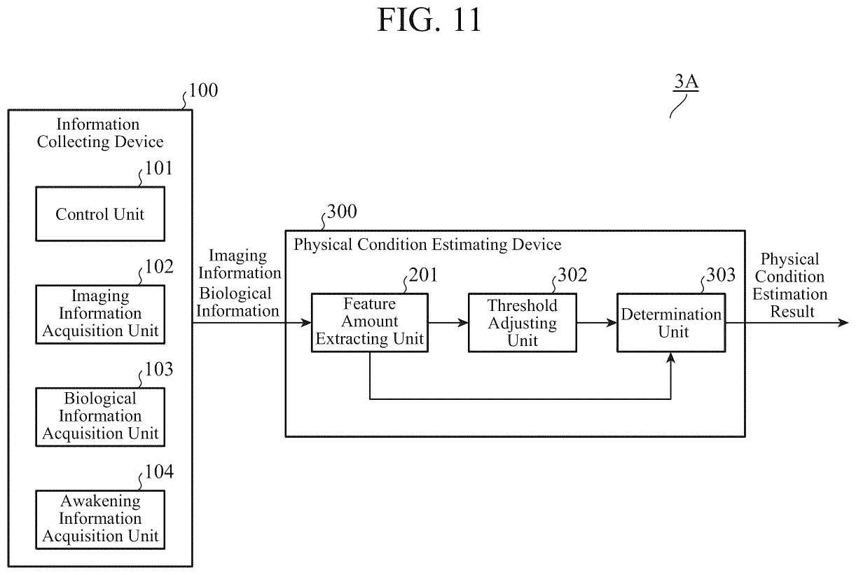

Next, a physical condition estimating system 3A according to the third embodiment will be described with reference to FIGS. 11 to 13. FIG. 11 is a block diagram illustrating the physical condition estimating system 3A according to the third embodiment. As illustrated in FIG. 11, the physical condition estimating system 3A according to the third embodiment includes the information collecting device 100 and the physical condition estimating device 300 according to the first embodiment. Although the physical condition estimating device 300 according to the third embodiment is different from the physical condition estimating device 200 according to the second embodiment in that a physical condition estimation result can be output without using a trained model, some configurations are similar to those of the physical condition estimating device 200 according to the second embodiment, and the configurations similar to those of the second embodiment are denoted by the same reference numerals and description thereof is omitted.

The physical condition estimating device 300 as an awakening level estimating device and a threshold setting device is a device that estimates the awakening level of a subject on the basis of the imaging information of the subject using the information collected by the information collecting device 100. As illustrated in FIG. 11, the physical condition estimating device 300 includes a feature amount extracting unit 201, a threshold adjusting unit 302, and a determination unit 303.

The threshold adjusting unit 302 as a threshold setting unit sets a threshold for estimating the awakening level of the subject using the feature amount extracted by the feature amount extracting unit 201. For example, when the feature amount extracted by the feature amount extracting unit 201 is a feature amount indicating a heartbeat, and it is determined that the awakening level of the subject has decreased when a change in the heartbeat for a predetermined time (for example, for 10 seconds) is equal to or more than 20 bpm on the basis of the feature amount, the threshold adjusting unit 302 sets 20 bpm as the threshold. Further, the threshold adjusting unit 302 adjusts the threshold set in advance to an appropriate value on the basis of the feature amount extracted by the feature amount extracting unit 201. For example, when the threshold for estimating the awakening level is set to a value for which estimation accuracy of the awakening level is not sufficient on the basis of the feature amount extracted by the feature amount extracting unit 201 and the information regarding the awakening level of the test subject related to the information from which the feature amount has been extracted, the threshold adjusting unit 302 adjusts the threshold to improve the estimation accuracy of the awakening level.

The determination unit 303 as an estimation unit determines the awakening level of the subject using the threshold set by the threshold adjusting unit 302. For example, in a case where the feature amount extracted by the feature amount extracting unit 201 from the imaging information or the biological information of the subject exceeds the threshold set by the threshold adjusting unit 302, the determination unit 303 estimates that the subject is in a state where the awakening level has decreased or a state where there is a sign of a decrease in the awakening level. The feature amount used for estimation by the determination unit 303 may be imaging information or biological information acquired from the information collecting device 100, or may be imaging information or biological information acquired from the imaging device or the biological information acquisition device (not illustrated). The determination unit 303 outputs the estimation result to the outside.

The physical condition estimating device 300 may include a processor, a memory, and an I/O port, and be configured in such a manner that the processor reads and executes a program stored in the memory, or may include a processing circuit and an I/O port that are dedicated hardware, and be configured in such a manner that the processing circuit executes the program. Since the hardware configuration of the physical condition estimating device 300 is similar to that of the information collecting device 100 according to the first embodiment, the description thereof will be omitted.

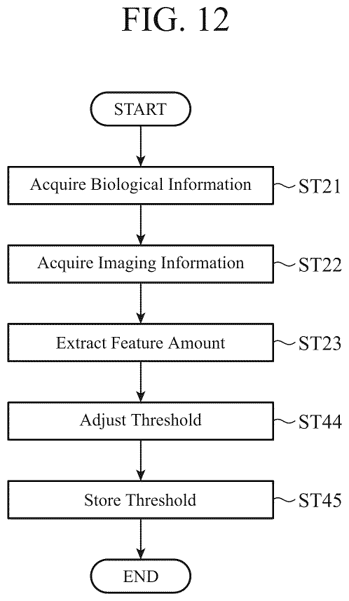

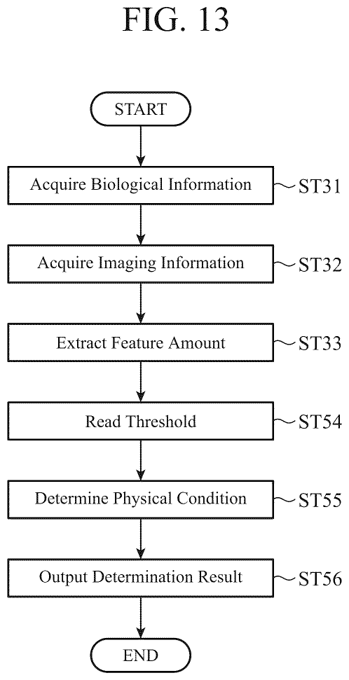

Next, processing related to the setting of the threshold performed by the physical condition estimating device 300 according to the third embodiment will be described with reference to FIGS. 12 and 13. FIG. 12 is a flowchart illustrating processing related to setting of a threshold performed by the physical condition estimating device 300 according to the third embodiment. First, when the processing is started, the physical condition estimating device 300 acquires the biological information collected by the information collecting device 100 (step ST21), and acquires the imaging information collected by the information collecting device 100 (step ST22). Note that the physical condition estimating device 300 may be configured to acquire information from the information collecting device 100 in a state of being electrically connected to the information collecting device 100 so as to be able to communicate information, or may be configured to acquire information from the information collecting device 100 via a recording medium.

After performing the processing of steps ST21 and ST22, the physical condition estimating device 300 extracts a feature amount from the acquired information (step ST23). After performing the processing of step ST23, the physical condition estimating device 300 sets a threshold for estimating the awakening level of the subject (step ST44). In this processing, in a case where the threshold is not set in advance, the threshold adjusting unit 302 sets a new threshold, and in a case where the threshold is set in advance, the threshold adjusting unit sets a new threshold by adjusting the threshold so as to be a more appropriate value. After performing the processing of step ST44, the physical condition estimating device 300 stores a new threshold in the storage unit (not illustrated) (step ST45).

Next, processing related to estimation of the awakening level performed by the physical condition estimating device 300 according to the third embodiment will be described by exemplifying processing in which the physical condition estimating device 300 estimates the awakening level of the subject on the basis of imaging information and biological information acquired from the imaging device and the biological information acquisition device (not illustrated). FIG. 13 is a flowchart illustrating processing related to estimation of the awakening level performed by the physical condition estimating device 300 according to the third embodiment. Note that, in the third embodiment, the determination unit 303 constitutes an imaging information acquisition unit that acquires imaging information including imaging of the face of the subject.

First, when the processing is started, the physical condition estimating device 300 acquires biological information of the subject from the biological information acquisition device (not illustrated) (step ST31), and acquires imaging information including imaging of the face of the subject from the imaging device (not illustrated) (step ST32). For example, the physical condition estimating device 300 acquires the biological information and the imaging information in a state of being electrically connected to the imaging device and the biological information acquisition device (not illustrated) so as to be able to communicate information. For example, the imaging device (not illustrated) is an imaging device that images an occupant of a vehicle, and the biological information acquisition device (not illustrated) is a sensor that is provided in the vehicle and acquires biological information of the occupant.

After performing the processing of steps ST31 and ST32, the physical condition estimating device 300 extracts a feature amount from the acquired information (step ST33). In this processing, the feature amount extracting unit 201 extracts a feature amount according to a set threshold value from the acquired information. For example, the feature amount extracting unit 201 extracts a feature amount regarding biological information of the test subject from the acquired imaging information.

After performing the processing of step ST33, the physical condition estimating device 300 refers to information stored in a storage unit (not illustrated) and reads a set threshold (step ST54). Note that, when the threshold adjusting unit 302 is configured to set a plurality of thresholds, the physical condition estimating device 300 may be configured to select one of the thresholds on the basis of the extracted feature amount.

When the processing of step ST54 is performed, the physical condition estimating device 300 estimates the awakening level of the subject by the determination unit 303 on the basis of the extracted feature amount and the threshold stored in the storage unit (step ST55). After performing the processing of step ST55, the physical condition estimating device 300 outputs information regarding the estimated awakening level of the subject and ends the processing (step ST56).

As described above, the physical condition estimating system 3A according to the third embodiment includes the threshold adjusting unit 302 that sets a threshold on the basis of the feature amount extracted from the imaging information including the imaging of the face of the test subject and the information regarding the awakening level of the test subject, and the determination unit 303 that acquires the imaging information including the imaging of the face of the subject, and the determination unit 303 estimates the awakening level of the subject on the basis of the threshold set by the threshold adjusting unit 302 and the acquired imaging information. Thus, it is possible to estimate the awakening level of the subject on the basis of the imaging information of the subject, and it is possible to manage the physical condition of the subject more easily than before.

Further, the physical condition estimating device 300 according to the third embodiment includes the feature amount extracting unit 201 that extracts a feature amount from the imaging information acquired by the information collecting device 100, and the threshold adjusting unit 302 that sets a threshold on the basis of the information regarding the awakening level of the test subject acquired by the information collecting device 100 and the feature amount extracted by the feature amount extracting unit 201. Thus, the physical condition estimating device 300 can estimate the awakening level of the subject on the basis of the threshold set on the basis of a sufficient amount of information acquired by the information collecting device 100, and can improve the reliability of the estimation result of the awakening level.

Note that, in the present disclosure, free combinations of the individual embodiments, modifications of any components of the individual embodiments, or omissions of any components in the individual embodiments are possible.

### INDUSTRIAL APPLICABILITY

An information collecting system according to the present disclosure can be used, for example, for production of a physical condition estimating device for estimating a physical condition of an occupant of a vehicle such as a car. Further, the physical condition estimating device according to the present disclosure can be used, for example, in a vehicle capable of estimating the physical condition of an occupant.

### REFERENCE SIGNS LIST

1A: information collecting system, 2A: physical condition estimating system, 3A: physical condition estimating system, 100: information collecting device, 101: control unit, 102: image information acquiring unit, 103: biological information acquisition unit, 104: awakening information acquisition unit, 200: physical condition estimating device, 201: feature amount extracting unit, 202: learning unit, 203: model storage unit, 204: state estimating unit, 300: physical condition estimating device, 302: threshold adjusting unit, 303: determination unit, B1: mounting table, B1a: mounting surface, C1: imaging unit, L0: center line, L1: imaging range, M1: drive unit, N1: input unit, P1: test subject, S1: sensor

1. An information acquisition device comprising processing circuitry
to acquire imaging information of a test subject placed on a mounting surface from an imaging device, which is held in such a manner that a position of the imaging device with respect to the mounting surface is fixed, to image the test subject, and
to acquire information regarding an awakening level of the test subject placed on the mounting surface, wherein
the processing circuitry acquires the imaging information including a face of the test subject placed on the mounting surface that moves between a first position where an angle between the mounting surface and a horizontal plane is a first angle and a second position where the angle between the mounting surface and the horizontal plane is a second angle.

2. The information acquisition device according to claim 1, wherein the processing circuitry is further configured
to acquire biological information of the test subject placed on the mounting surface.

3. The information acquisition device according to claim 1, wherein
the angle with a horizontal plane in the second position is larger than the angle with a horizontal plane in the first position, and
the processing circuitry acquires the imaging information including at least the face of the test subject placed on the mounting surface at a certain timing after a timing at which a position of the mounting surface changes from the first position to the second position.

4. The information acquisition device according to claim 1, wherein
the processing circuitry acquires the imaging information of the test subject placed on the mounting surface in a state in which the mounting surface is located at the second position, a state in which the mounting surface is moving from the first position to the second position, a state in which the mounting surface is moving from the second position to the first position, and a state in which the mounting surface is located at the first position in such a manner that the imaging information acquired in the state in which the mounting surface is located at the second position or in the state in which the mounting surface is moving from the first position to the second position, and the imaging information acquired in the state in which the mounting surface is located at the first position or in the state in which the mounting surface is moving from the second position to the first position are the imaging information in a same range with respect to the mounting surface.

5. The information acquisition device according to claim 1, wherein the processing circuitry is further configured
to control the angle between the mounting surface and the horizontal plane in such a manner that the mounting surface moves between the first position and the second position.

6. A learning device comprising processing circuitry
to acquire imaging information of a test subject placed on a mounting surface from an imaging device, which is held in such a manner that a position of the imaging device with respect to the mounting surface is fixed, to image the test subject,
to acquire information regarding an awakening level of the test subject placed on the mounting surface,
to extract a feature amount from the imaging information, and
to generate a trained model on a basis of the information regarding the awakening level of the test subject and the feature amount, wherein
the processing circuitry acquires the imaging information including a face of the test subject placed on the mounting surface that moves between a first position where an angle between the mounting surface and a horizontal plane is a first angle and a second position where the angle between the mounting surface and the horizontal plane is a second angle.

7. An information acquisition method performed by a device including processing circuit, the information acquisition method comprising:
acquiring imaging information of a test subject placed on a mounting surface from an imaging device, which is held in such a manner that a position of the imaging device with respect to the mounting surface is fixed, to image the test subject; and
acquiring information regarding an awakening level of the test subject placed on the mounting surface, wherein
the processing circuitry acquires the imaging information including a face of the test subject placed on the mounting surface that moves between a first position where an angle between the mounting surface and a horizontal plane is a first angle and a second position where the angle between the mounting surface and the horizontal plane is a second angle.
